(19) United States
(12) Patent Application Publication
(10) Pub. No.: US 2026/0345003 A1
(43) Pub. Date: Nov. 5, 2026

(54) SYSTEMS AND METHODS FOR GENERATING SIMULATED COLOR PHOTOGRAPHIC IMAGES FROM OPTICAL COHERENCE TOMOGRAPHY DATASETS

(71) Applicant: Oregon Health & Science University, Portland, OR (US)

(72) Inventors: Benjamin K. Young, West Linn, OR (US); Yifan Jian, Portland, OR (US); John Peter Campbell, Lake Oswego, OR (US); Yakub Bayhaqi, Portland, OR (US); David Sutter, Portland, OR (US); David Huang, Portland, OR (US)

(21) Appl. No.: 19/666,205

(22) Filed: May 2, 2026

Related U.S. Application Data
(60) Provisional application No. 63/799,335, filed on May 2, 2025.

(51) Int. Cl. A61B 3/14; A61B 3/00; A61B 3/10; A61B 3/13

(52) U.S. Cl. CPC A61B 3/14; A61B 3/0025; A61B 3/102; A61B 3/13

(57) ABSTRACT

Systems and methods for generating color images from near-infrared optical coherence tomography datasets are described. In various embodiments, knowledge of the expected tissue interaction with visible light wavelengths is used to generate a set of blue, green, and red channel images which, when combined, produce a color image. Methods are provided for generating the required blue, green, and red channels data using a wavelength-dependent model to simulate the attenuation and reflection of incident light as it passes through an optical coherence tomography dataset. Also provided are methods wherein an optical coherence tomography dataset is segmented into a plurality of retinal and choroidal slabs which are processed to provide weighted contributions to each of the blue, green, and red color channels. In addition, a segmentation-free technique is described for generating a color image based on the weighted contributions of various en face projection images derived from an optical coherence tomography dataset.

### CROSS-REFERENCE TO RELATED APPLICATIONS

This application claims the benefit of U.S. provisional patent application No. 63/799,335, filed May 2, 2025, titled “Systems and Methods for Generating Simulated Color Photographic Images from Optical Coherence Tomography Datasets,” which is incorporated herein by reference.

### ACKNOWLEDGEMENT OF GOVERNMENT SUPPORT

This invention was made with government support under R01 HD107494 awarded by The National Institutes of Health. The government has certain rights in the invention.

### TECHNICAL FIELD

This disclosure relates to systems and methods of using structural volumetric optical coherence tomography (OCT) and knowledge of the expected tissue interaction with visible light to generate simulated color photographic images of the retina, analogous to images obtainable by fundus photography or scanning laser ophthalmoscopy.

### BACKGROUND INFORMATION

Optical coherence tomography (OCT) is the most commonly used retinal imaging device globally, using coherent near-infrared light to obtain micrometer-level depth resolved images of biological tissue based on variable tissue reflectivity. OCT has revolutionized retinal diagnostics, becoming the most commonly used imaging modality in the ophthalmology clinic. Its unparalleled ability to obtain non-invasive, high resolution volumetric clinical images has surpassed clinical exams, and allows for objective monitoring of disease, guiding treatment and decision making. However, as OCT relies on near infrared laser interferometry, it still falls short of clinical examination in being able to perceive color, which can have critical implications in retinal diagnostics. Therefore, standard of care remains to perform clinical ophthalmoscopy to produce color fundus photos for complete retinal examination, supported by fundus photography or scanning laser ophthalmoscopy.

However, it is increasingly common for patients in retina clinics to be imaged only by an OCT device, as separate acquisition of color fundus photos can be time-consuming or impractical. Further, acquisition of fundus photographs requires that the patient's pupil be dilated, which is an unpleasant experience for some patients and causes blurry vision for many hours after examination. All currently available techniques to produce color fundus images require bright, visible light sources, which may also be uncomfortable for patients, and can sometimes make imaging impossible in children, or developmentally delayed patients. Thus, there is a need for alternate methods to generate color fundus photos in a way that streamlines workflow and minimizes patient discomfort and inconvenience.

### SUMMARY OF THE DISCLOSURE

Systems and methods are disclosed for inferring the red, green, and blue reflectance of tissue using near-infrared OCT datasets and knowledge of the expected tissue interaction with visible light to generate color OCT images. When used in conjunction with OCT systems and devices capable of acquiring wide field and ultra-wide field images, the systems and methods disclosed herein may be used to generate color images similar to those obtainable using color fundus photography or scanning laser ophthalmoscopy.

In some embodiments, the systems and methods may comprise receiving or acquiring an OCT dataset of an imaged retina and choroid and simulating the attenuation and reflection of a photon packet as it passes stepwise in the depth direction through the pixels making up the A-scans of the OCT dataset. For each A-scan, a total reflected signal accumulated from each photon packet and pixel interaction. This total reflected signal is calculated for each of a blue, green, and red wavelength packets having progressively smaller reflectivity characteristics, and then assigning each of the calculated total reflected signals to a corresponding blue, green, and red channel. The resultant blue, green, and red channels are then merged or combined to generate a color image. In some embodiments, the pixel values of the blue, green, and red channels may be normalized or weighted prior to merging.

In an alternate embodiment, systems and methods are provided for obtaining an OCT dataset of the posterior eye and segmenting it into three or more volumetric slabs. In some embodiments, the segmented slabs may include the retinal nerve fiber layer (RNFL), retinal pigmented epithelium (RPE), the retina or a portion thereof, and/or the choroid or a portion thereof. These segmented slabs are used to create a set of en face projection images from which a blue, green, and red channel are populated, wherein each of the channels is comprised of a weighted contribution from the set of en face projection images. These channels are then merged to generate a final color image.

Also provided are systems and methods for segmentation-free generation of a color image from an OCT dataset. In this approach a set of two or more en face projection images are generated from the OCT dataset and used to populate, a green channel, a red channel and, optionally, a blue channel. Population of the channels may utilize a weighted contribution from the one or more of the en face projection images. In a particular embodiment, one of the en face projection images is a mean intensity projection image and another of the en face projection images is a maximum intensity projection image.

Additional aspects and advantages will be apparent from the following detailed description of preferred embodiments, which proceeds with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Applicant considers the color versions of the drawings as part of the original submission and reserves the right to present color images of the drawings in later proceedings.

To easily identify the discussion of any particular element or act, the most significant digit or digits in a reference number refer to the figure number in which that element is first introduced.

FIG. 1 shows an example of a typical multicolor scanning laser ophthalmoscopy image, with the blue, green and red channels separated, along with a B-scan illustrating the relative depth of penetration of blue, green, and red wavelengths of light.

FIG. 2A shows a set of choroid, retina, and RNFL mean-intensity en face projections constructed from segmented an UWF-OCT dataset. Contributions from the choroid, retina, and RNFL en face layers were projected into red, green, and blue color channels to generate a simulated color photographic image of the retina.

FIG. 2B shows a schematic example of the relationship between the variable penetrance of visual light blue, green, and red components into retinal and choroidal tissue of a representative B-scan, and the associated segmented OCT en face image slabs at successive tissue depths.

FIG. 3 shows an example of a simulated color fundus image generated using UWF-OCT image data taken from an infant with retinopathy of prematurity (left), along with an actual fundus photo taken the same day (right).

FIG. 4 shows an example of a color image generated using a weighted blending of blue, green, and red channels (left) and a color image generated using only green and red channels (right).

FIG. 5 schematically shows an example system for generating color images from OCT datasets in accordance with the disclosure.

FIG. 6 schematically shows an example of a computing system in accordance with the disclosure.

### DETAILED DESCRIPTION OF EMBODIMENTS

Disclosed herein are systems and methods to infer color information for retinal and choroidal tissues from structural OCT signals based on the expected interaction of visible light with those tissues. The disclosed systems and methods take advantage of the fact that visible light has variable penetration through the various retinal and choroidal layers. Accordingly, the systems and methods described herein may be used to generate simulated color photos from near-infrared structural OCT reflectance data. In particular, when OCT data is acquired using ultra-widefield OCT systems, such as a contact-based OCT system used in the screening of retinopathy of prematurity or a non-contact experimental OCT device as described in the literature, simulated color photographic images equivalent to those obtained by fundus photography or scanning laser ophthalmoscopy may be generated.

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use the terms “embodiment” or “embodiments,” which may each refer to one or more of the same or different embodiments. Furthermore, the terms “comprising,” “including,” “having,” and the like, as used with respect to embodiments, are synonymous.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

All publications, patent applications, patents, and other references mentioned herein are incorporated by reference in their entirety. In case of conflict, the present specification, including explanation of terms, will control. In addition, the methods, systems, apparatuses, materials, and examples are illustrative only and not intended to be limiting.

In order to facilitate review of the various embodiments of the disclosure, the following explanation of specific terms is provided:
A-scan: A reflectivity profile that contains information about spatial dimensions and location of structures within an item of interest. An A-scan is directed along the optical axis of the OCT device and penetrates the sample being imaged. The A-scan encodes reflectivity information (for example, signal intensity) as a function of depth. The digitized version of an A-scan as used herein is understood to comprise a set of pixels ordered in the depth direction along the optical axis of the OCT device, with each pixel storing a signal value. When the term “reflectance” is used herein, it generally refers to the fraction of light reflected from the eye along an A-scan as measured by an OCT device.
B-scan: A cross-sectional tomograph that may be achieved by laterally combining a series of axial depth scans (i.e., A-scans). A B-scan encodes planar cross-sectional information from the sample and is typically presented as an image comprised of an ordered array of pixels with each pixel associated with a signal value.
OCT Dataset: As used herein, an OCT dataset is an ordered-array representation of stored data values that encodes relative spatial location in row-column-depth (x-y-z) format. In the context of OCT, as used herein, a dataset may be conceptualized as a three-dimensional array of pixels (pixels are often referred to as “voxels” when presented in a three-dimensional or volumetric context), each pixel having an associated value (for example, an intensity value or a reflectance value). An A-scan corresponds to a set of collinear pixels in the depth (axial or z) direction of the dataset; a B-scan is made up of set of adjacent A-scans combined in the lateral (row) direction. A collection of adjacent B-scans can be combined in the vertical (column) direction to form a 3D volumetric set of pixel data (or 3D image comprised of voxels). The most basic form of an OCT dataset as used herein is a single A-scan.
Depth direction: as used herein, in the context of OCT datasets of posterior pole of the eye, the terms “depth direction,” and “z-direction”, refer to a direction that is parallel to the direction extending from the vitreous, into and through the retina, an onward into and through the choroid. Thus, the depth direction is parallel to (or extends along) an A-scan produced by an OCT beam incident on the retina.
en face image: OCT data can be presented as a projection of the three dimensional dataset onto a single planar image called an en face image (Wallis J et al, Med Imaging IEEE Trans 8, 297-230 (1989); Wang R K et al, Opt Express 15, 4083-4097 (2007); Jia Y et al, Opt Express 20, 4710-4725 (2012)); incorporated by reference herein). Construction of such an en face image requires the specification of the upper and lower depth extents that enclose the region of interest within the OCT scan to be projected onto the en face image. These upper and lower depth extents can be specified as the boundaries between different layers of the retina (e.g., the pixels between the retinal nerve fiber layer and retinal pigment epithelium can be used to generate a 2D en face image of the retina). The pixel values of an A-scan can be projected onto a single pixel having a single value. A set of A-scans comprising a volume and their associated signal values can be projected to form a two dimensional set of single-valued pixels (e.g., an image comprised of pixels, or a single channel of a color image, such as a red channel, green channel, or blue channel of an RGB image). In the context of the present disclosure, an en face image may alternately be referred to as en face projection image, an en face projection, or projection image. Typical examples of en face projection include mean intensity projection, maximum intensity projection, and summed intensity projection.
Segmentation: As used herein, the term “segmentation,” and its equivalents, can refer to a process of dividing an image or volume of a retina/choroid OCT dataset into regions or volumes (i.e., sub-volumes within the full volumetric OCT dataset) corresponding to different retinal layers, anatomic regions, or portions of anatomic regions. For instance, a segmentation method can be performed by defining the positions of boundaries between an inner and outer layer of the retina to define a 3D volume of the retina which excludes the choroid. Throughout the present disclosure, a volumetric segmentation of the retina, choroid, or portions thereof, is referred to as a “slab.” A typical volumetric slab produced by a segmentation operation is comprised of a plurality of A-scans. Such a slab may be projected to form an en face image, as described above. It is also noted that, in the context of the present disclosure, a slab may sometime a single pixel thick, such as a slab of the retinal pigment epithelium.
Inversion: as used herein, inversion is defined as identifying the maximum pixel value in a set of pixels (assuming that the minimum pixel value is zero), then subtracting current pixel values from this maximum value to calculate the inverted pixel intensity. Inversion may be applied, for example, to an A-scan, an image, a color channel, or a slab. When applied to an image comprised of grayscale pixel values, for example, the inverted image maintains the same relative spatial arrangement of its constituent pixels, but the “scale” is reversed such that darker pixels from the original image appear lighter and lighter pixels appear darker.

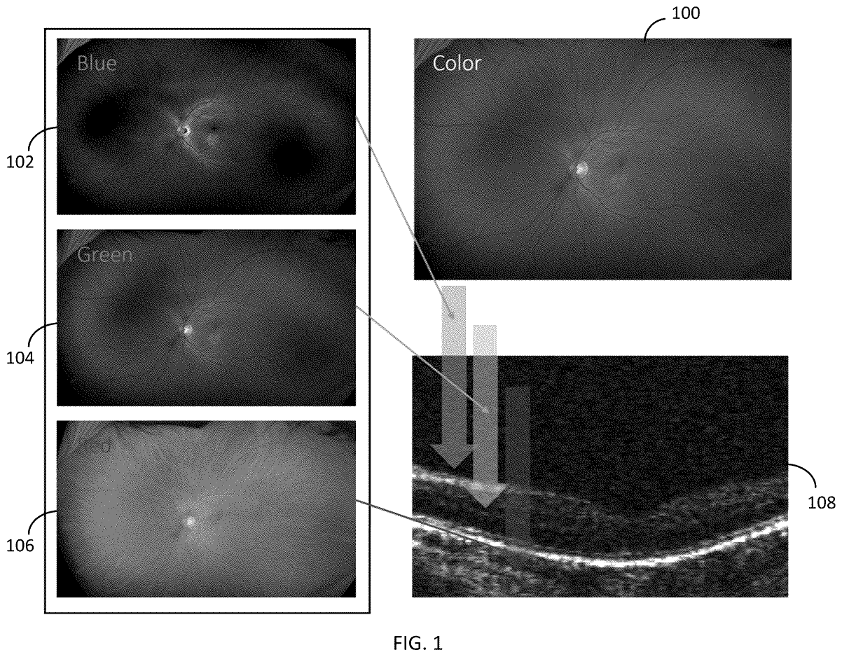

FIG. 1 shows an exemplary color scanning laser ophthalmoscopy photo 100, with the color photo separated individual images for the blue channel 102, the green channel 104 and the red channel 106 channel. Blue, green and red wavelengths of light have variable penetration into the retinal and choroidal tissue, owing to fundamental properties of the varying biological tissue layers comprising the retinal and choroid. The inset in the lower right of FIG. 1 shows a sample OCT slice (B-Scan) 108, and schematically depicts the variable penetrance of the blue, green and red wavelengths into and through the different layers of the retinal and choroidal tissue. As depicted by arrows, blue wavelength light penetrates the more superficial layers of the retina, green wavelength light penetrates to an intermediate depth within the retina, and red wavelength light penetrates deepest through the full thickness of the retina and into the choroid.

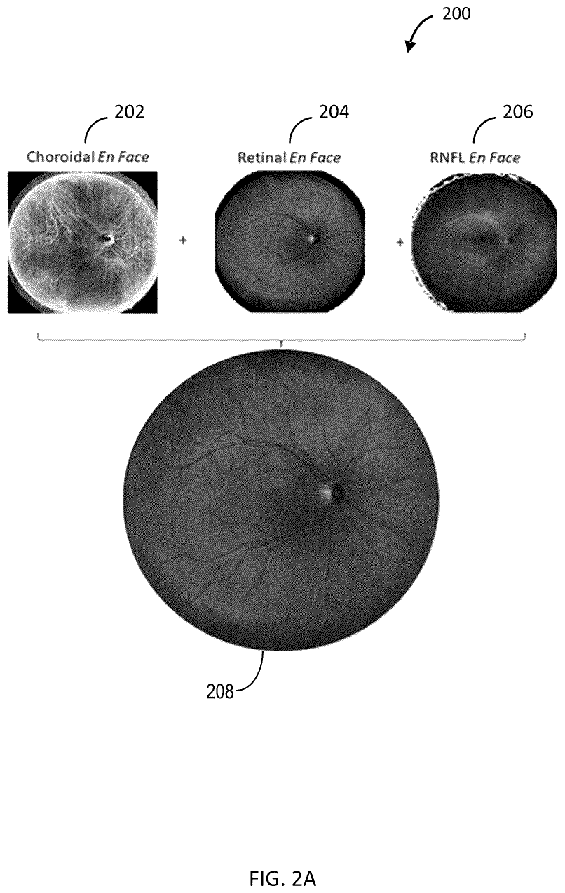

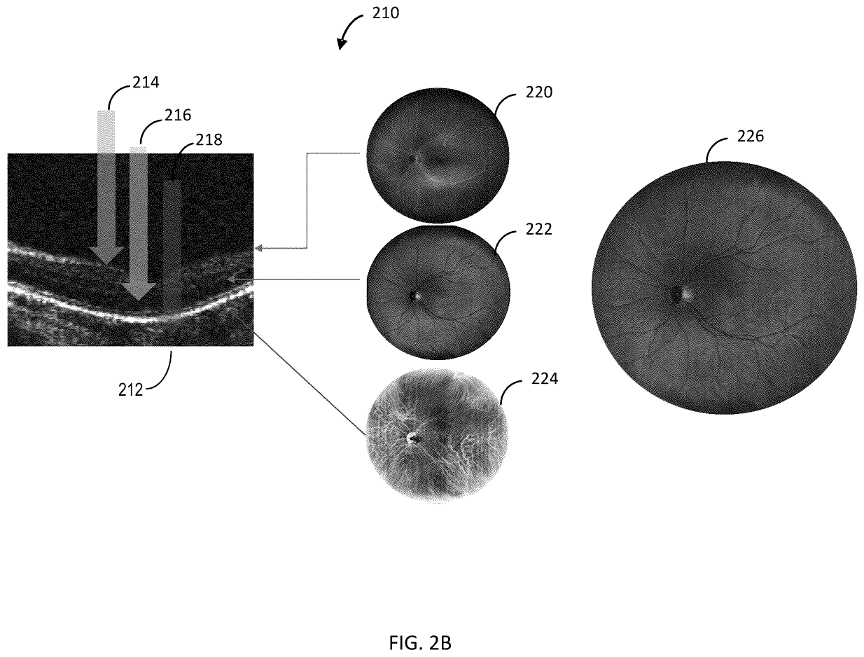

FIG. 2A shows a visual summary 200 of an embodiment of the methods disclosed herein for generating a simulated color fundus photo from an OCT dataset. As shown, three “slab” subvolumes are segmented from an OCT dataset and presented as en face mean intensity projection images: the choroid 202, retina 204, and retinal nerve fiber layer (RNFL) 206. In the particular embodiment of the method summarized here, to produce a simulated color fundus image 208, each of these three layers is assigned weighted contributions to the red, green, and blue color channels, then summed together to produce a realistic color retinal image 208.

As part of the processing of the OCT data to generate a simulated color fundus image 208 such as the one shown in FIG. 2A, the choroidal slab 202 underneath the retinal pigmented epithelium (RPE) is inverted, taking advantage of the fact that high blood flow through the choroid appears dark on OCT, so inversion makes the choroidal flow red. Therefore, features which cast shadows on the choroid will appear red in the red color channel after inversion. Further, retinal blood flow overlying the choroid blocks the light signal passing through the tissue and casts a “dark shadow” on the choroid. Therefore, features which cast shadows on the choroid will appear red in the red color channel after inversion Upon inversion, the retinal blood flow within the tissue overlying the choroid also appears red.

In additional processing steps, the layers comprising the “retinal en face” image slab 204 are added to both the red and green channel. This is done because the retinal en face slab includes the RPE (retinal pigment epithelium), the brightest and most reflective layer of the retina. The RPE is primarily composed of melanin, which is brown in pigmentation, and composed of similar proportions of red and green. Finally, while blue is the weakest contributor to color fundus photos, it has been observed that the more superficial structures in the retina reflect blue wavelength light. Therefore, a superficial segmented slab encompassing the retinal nerve fiber layer (RNFL) 206 (denoted in FIG. 2A as the “RNFL en face” image slab) is added to the blue channel. Together, these combined red, green, and blue channels allow simulated color fundus photos to be generated, solely from the infrared OCT reflectance signals.

FIG. 2B shows another summarized example 210 of the relationship between the variable penetrance of visual light spectral components into retinal and choroidal tissue and the associated segmented OCT slabs used to generate a simulated color fundus image. Again, as shown schematically on a representative B-scan 212, blue wavelength light 214 exhibits superficial penetration of the retina, green wavelength light 216 penetrates the retina to an intermediate depth, and red wavelength light 218 penetrates deeply through the thickness of the retina and into the choroidal space. A superficial slab 220 encompassing the RNFL is segmented from the structural OCT dataset to correspond with the penetrance of the blue wavelength light 214 to populate the blue channel. An intermediate slab 222 encompassing the RPE is segmented to correspond with the primarily green wavelength light 216 to populate the green channel. A deepest slab 224 encompassing the choroid is segmented to correspond with the penetrance of the red wavelength light 218 to populate the red channel. Note that the green channel may also include partial contribution from the deepest slab where the red wavelength light is predominant. When the populated red, green, and blue channel data are merged or added, a simulated color photographic image 226 is produced.

FIG. 3 shows an example of a simulated color fundus image 300 generated using UWF-OCT image data (left) taken from an infant with retinopathy of prematurity, along with a fundus photo 302 taken the same day (right). In this figure, simulated color fundus image 300 was produced in the manner outlined in the visual summary 200 of FIG. 2A and associated description. It is noted that the small, comma-shaped hemorrhage on the right side of the simulated fundus image 300 appears red without any special tuning or scaling to force it to turn red. This example demonstrates that the disclosed methods can be used to highlight pathology, such as hemorrhage as shown here, that OCT imaging has classically been described as not being able to visualize well.

As another example of a retinal pathology amenable to analysis the disclosed methods, exudative age-related macular degeneration is a condition characterized by either retinal edema, or retinal hemorrhage. Classically, OCT is by far the best modality to detect retinal edema, but standard of care for retinal specialists is to still perform routine dilated fundus examinations to look for intraretinal hemorrhages that OCT cannot detect. The methods and systems disclosed herein can obviate the need for such uncomfortable dilations and eye exams for patients and instead allow retina specialists to streamline workflow by generating simulated color fundus images based on OCT image data alone.

It will be appreciated by those having skill in the art that alternate approaches may be used to infer the interaction of red, green, and blue light with the tissue structures imaged by the near-infrared OCT signal. Accordingly, several methods are outlined below for assigning OCT signal intensity contributions from various slabs of the OCT dataset to red, green and blue channels to generate a combined RGB simulated color photographic image from OCT data. It is to be understood that the methods described below are intended to be exemplary and are not intended to limit the scope of the disclosure.

In some embodiments, the disclosed systems and methods may utilize a simulation-based modeling approach to generate a color OCT image from an OCT dataset. In a particular implementation a set of simulated blue, green and red wavelength “packets of light” are each passed through the OCT dataset along the individual A-scans comprising the OCT dataset. As a packet of light proceeds stepwise through a given A-scan, the interaction of the light packet with consecutive pixels representing the retinal and choroidal tissues causes a progressive attenuation of the amount of photons in the light packet. That is, the intensity of the blue, green, or red light beam incident upon the retina diminishes as the light beam traverses the depthwise through the A-scan. As a working simplification, this simulation may be constructed by assuming that as light interacts with tissue, it will either: a) reflect, b) absorb, c) scatter, d) transmit (and thus is progressively attenuated). Each wavelength of light (blue, green, red) is characterized by a reflectivity parameter that governs the percentage of light reflected backward along the A-scan to the OCT device detector as the light interacts with successive tissue pixel intensity values along the forward direction of the A-scan. It is assumed in the simulation that this reflected light is lost from the intensity of the package of photons, and that a further loss occurs from inferred absorption and scatter (the simulation may be further simplified by assuming the scattered light is simply lost). The simulation continues step-wise through successive pixels along the A-scan and continues this process until either all photons are lost, or the bottom of the scanning image is reached. The amount of light reflected back along the A-scan for each pixel is accumulated (summed) to calculate a total reflected signal value for that traversal. This simulation process is performed for all A-scans of the OCT dataset, and the total reflected signal value for each A-scan is assigned to a corresponding pixel in the corresponding blue, green, or red channel. It is noted that assignment of total reflected signal value to a given color channel is analogous to the generation of an en face projection image in that channel.

In this simulation scheme, red, green and blue wavelength light contributions to their respective color channels are each governed by reflectance, scatter, and transmission parameters appropriate to their known physical properties. In some embodiments, a wavelength-dependent reflectance parameter may be specified (as noted above), and the scatter and transmission parameters may then be defined as multiples or fractions of the specified reflectance parameter. Specification of the reflectance parameter for each wavelength of light is guided by its observed interaction with tissues of the posterior eye: blue is initially highly reflective, but is then quickly absorbed or scattered in the tissue; green is somewhat less reflective, but has less absorption and scattering than blue, allowing it to penetrate deeper into the tissue; and red is less reflective, but also has less absorption/scattering, allowing it to penetrate most deeply into the tissue.

It is also necessary in this simulation scheme to invert the intensity of the reflected signal at the level of the RPE and below (i.e., the choroid), but only for the red photon packages. In practice, this may be achieved by segmenting the choroidal slab from the volumetric OCT dataset and inverting the pixel values. The reasoning for this is that as heme reflects red light much more than green or blue light (which get absorbed by heme), the abundance of heme in the choroidal vessels which are dark on OCT would be bright in the red channel, necessitating this inversion. Further, the retinal vasculature (or other heme, such as sub, intra or pre-retinal heme) blocks signal, causing shadowing against the RPE. Therefore, an inversion of the choroid and RPE signal itself will cause the red heme to appear brighter in the red channel itself.

Notably, this photon packet simulation technique can be applied to the OCT B-scans (i.e., cross sectional scans/images) as well, providing a simulated color of the retinal layers in cross section, and potentially allowing for 3D visualization of the retina in color.

The following paragraphs provide a further exemplary description of the simulation process for generating a color image from an OCT dataset. Beginning with the blue channel, the pixel intensity values to be assigned to the blue en face image are calculated by simulating the passage of a package of photons through each A-scan. In this example it is assumed that this package of photons has an initial value of 1.0. The pixel intensity values of the A-scan are normalized. A simulation is performed wherein the blue reflectivity parameter is specified such that X percentage of incident light proportional to a given pixel intensity is reflected back along the A-scan. This value is stored as blue channel pixel intensity. Next, that same value is subtracted from the package of photons twice over. This subtraction is performed twice in the present example, under the assumption that the quantity of light that is absorbed or scattered by the tissue is approximately equal to the quantity of light reflected (i.e., the intensity of the packet of light is attenuated (reduced) 1.0-2×). This updates the current value of the simulated package of photons, and this attenuation process continues for each depth position along the A-scan, until the simulation has progressed through the entire A-scan, or the intensity value of the simulated package of light becomes zero. The total accumulated value of reflected light is recorded for this particular A-scan, and the process is repeated for all remaining A-scans in the OCT dataset.

The above process is repeated for the green channel, where the green light penetrates more deeply than for blue. This is modeled by specifying a green reflectivity parameter that results in a smaller Y percentage of incident light being reflected back. For example, the green reflectivity parameter may be specified such that Y is about 80% of X as above (that is, the green reflectivity parameter is smaller than the blue reflectivity parameter). This allows the light of the green channel to penetrate deeper into the tissue and reflect deeper structures.

The above simulation process is then repeated for the red channel, but with two modifications. First, the percentage reflectance is reduced even further than for blue, approximately 50% of the value for the blue channel (that is, the red reflectivity parameter is smaller than the green) reflectivity parameter. Second, the pixel intensity for each A-scan posterior to the segmented retinal pigment epithelium layer (RPE) is inverted (which entails segmenting the pixels posterior to the RPE into a slab to apply the inversion operation). This inversion is performed to account for the fact that the choroidal structures are primarily vascularized, so that blood within the choroid, which possesses hemoglobin, appears dark on the A-scan, but should appear bright on the red channel during simulation. Further, it is observed that blood filled structures in the retinal layers, such as retinal blood vessels, can cause posterior shadowing of pixel intensity. Thus, the inversion of the layers posterior the retinal pigment epithelium will appear bright, reflecting the red color of the overlying blood-filled structure.

After each color channel is constructed using the simulation process detailed above, each pixel intensity may be normalized to a range of values from 0 to 1. Lastly, the blue, green and red channels (now represented as blue, green, and red en face images) are combined. to create a color image.

In an alternate embodiment, the disclosed systems and methods may be used to segment the retina and choroid into separate slabs, from which en face projection images may be derived and used to populate blue, green, and red channels to form a final colored image. In such implementation, a first slab encompassing the superficial portion of the retina inclusive of the retinal nerve fiber layer (RNFL), a second slab encompassing the retina exclusive of the RNFL, retinal pigmented epithelium (RPE), a third one-pixel wide “slab” of the RPE, and a fourth slab encompassing the choroid. Each of these slabs, or a portion thereof, may be used to generate an en face projection image from its constituent pixel intensity values. The en face projection images may be generated by mean intensity projection, maximum intensity projection, summed intensity projection, a weighted combination of these projection types, or other formulations. In addition, in some cases, a specified subset of the A-scan pixel intensity values within a given slab may be more heavily or lightly weighted in the generation of an en face projection image. In a typical application, it is often desirable to further normalize the pixel intensity values of the generated en face projection images.

Once generated, the set of generated en face projection images are used to populate blue, green, and red channel (i.e., one or more en face projection images may be added to a given blue, green, or red channel to produce a blue, green, or red channel image). In a typical implementation, the first slab (which includes the RNFL) is used to populate the blue channel; the second slab (which includes the RPE) is used to populate the green channel, and the third slab (which includes the choroid) is used to populate the green channel. In some implementations, a given channel may receive contributions from (i.e., be populated with) multiple en face projection images derived from two or more slabs or slab portions. These contributions may be weighted as required to achieve a particular color composition in the final image. Once the blue, green, and red channels are populated, they are merged (or added) to generate a single color OCT image.

The following paragraphs describe an exemplary set of steps to generate a color OCT image from a 3D volumetric OCT dataset that has been segmented into a first, second, and third slab encompassing, respectively, the RNFL, retina, RPE, and choroid, as described above. It is noted that this example is intended to be representative but is not intended to limit the scope or variations of procedural steps that may be practiced in the context of this disclosure, nor is it intended to prescribe constraints on the order in which various operations or steps are executed. Uniform reflectivity is assumed through slabs of the volumetric OCT dataset with respect to each visible wavelength color (red, green, and blue):
1) First, the choroidal slab (i.e., fourth slab) is inverted, with the reasoning as above that high flow blood appears dark in the choroidal layer; further, overlying blood in the retinal vasculature would also cause shadowing in the choroidal layer. This inverted choroidal slab is then summed along the A-scan direction (to generate an en face projection image), normalized and added to the red channel.
2) Next, the RPE slab (i.e., the third slab, which is one pixel thick) is added in even proportions (i.e., ⅓^{rd} signal) to each value to the red, green and blue channels. The motivation is that RPE's coloration is predominantly from melanin, or more specifically eumelanin, which is dark brown in coloration. As dark brown is a combination of red, green and blue, the RPE slab is then added to each of these channels.
3) Next, to simulate the green channel, the retinal slabs, plus a superficial portion of the choroidal slab (without inversion) are summed along the A-scan direction, with the deep retinal layers (i.e., the outer 50% of the pixels in the second slab) were further emphasized by doubling their contribution to this summation. This is then normalized and added to the green channel.
4) Finally, a superficial retinal layer (chosen as the inner 20% of pixels in the first slab) is summed along the A-scan direction (to generate an en face projection), normalized, and added to the blue channel, which again corresponds to blue light's shallow penetrance of retinal tissue.

In a further embodiment, the disclosed systems and methods may be applied in a manner that does not require the segmentation of different anatomic layers into slabs, but instead operates on the full 3D volumetric dataset. This approach may be advantageous, for example, in cases where segmentation is computationally prohibitive, or not available for a particular OCT platform. The underlying principle of this method is that the mean intensity projection of 3D volumetric data includes a combination of choroidal and retinal data, as it is a blended mean of all data along the Z axis of each A-scan. Further, it is observed that the maximum intensity projection closely represents the retinal data, as the maximum intensity pixel for each A-scan tends to localize with the RPE (retinal pigment epithelium).

Accordingly, a mean intensity projection of the 3D volumetric dataset is calculated along the Z axis (i.e., in the direction of the A-scans) to generate an en face mean intensity image. In practice, it may be desirable to further apply an auto-contrast to this en face image by calculating the 5^{th} and 99^{th} percentile intensity pixels, and clipping all those above and below these values. The remaining pixel intensity values in the en face image are then normalized to values from 0-1.

Similarly, a maximum intensity projection of the 3D volumetric data is calculated along the Z axis to generate an en face maximum intensity image. Because maximum intensity projections are prone to noise, it may be desirable to smooth the en face maximum intensity image by calculating the mean of the top 0.5% intensity pixels at each position. An auto-contrast operation may then be applied to the pixel values of the en face maximum intensity image, and then normalization applied as above.

A color image is then generated by blending the en face mean intensity image and the en face maximum intensity image in a weighted manner across the blue, green, and red channels to create an RGB color image. An example of such a weighted blending is as follows:

Blue Channel=Iₘₐₓ*w1

Green Channel=Iₘₐₓ*w2+Iₘₑₐₙ*w3

Red Channel=Iₘₑₐₙ*w4

where Iₘₐₓ is the en face maximum intensity image, Iₘₑₐₙ is the en face mean intensity image, and w1, w2, w3, and w4 are a set of weights specified such that w1, w2+w3, and w4 should not exceed 1. In typical use cases, the set of weight values may fall in the following ranges: w1=0.3-0.5; w2=0.4-0.6; w3=0.1-0.3; w4=0.9-1.0.

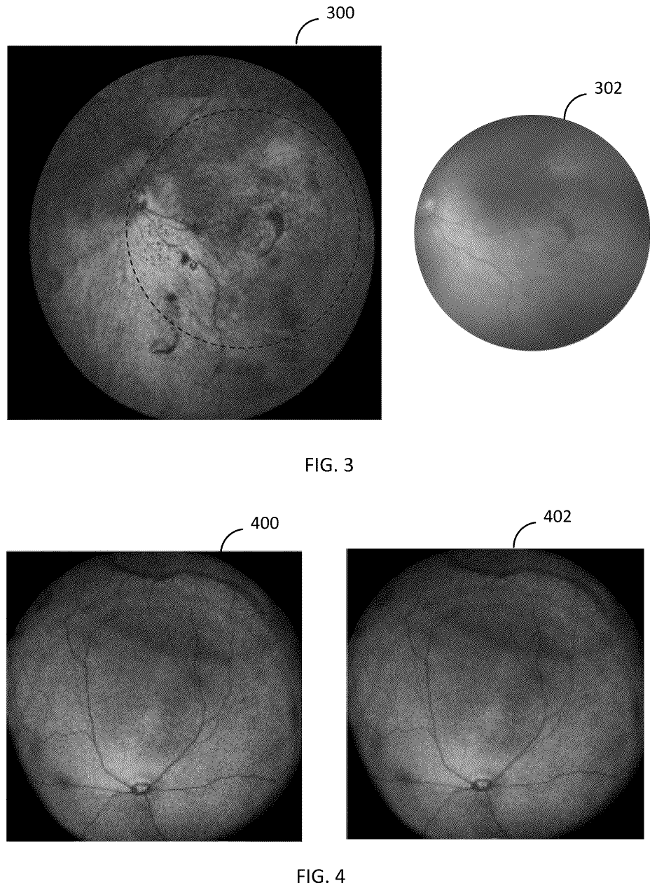

It should be noted that in some cases, it may be desirable to omit the blue channel from the blending operation (i.e., by setting w1=0) to better simulate the appearance of Red-Green scanning laser ophthalmoscopy images, such as the commercially available Optos California camera. FIG. 4 shows a set of example images illustrating the effect of omitting the blue channel example. The colorized OCT image 400 on the left was generated using the blue, green and red channels, while the image 402 on the right was generated using only the green and red channels.

### Example 1

Purpose: It is demonstrated that depth encoded data obtained in volumetric optical coherence tomography (OCT) can be used to reconstruct expected tissue interaction with visible light, producing simulated color retinal photographs.
Methods: Given that visible light spectrum has variable penetrance and reflectivity in the retinal layers (see FIG. 1 above), we used the mean intensity projection of different layers of the retina and choroid and projected their en face representation into differing color channels. The red channel was constructed from the choroid and partially from the retina, the green channel from the retina, and the blue channel from the retinal nerve fiber layer (RNFL), when available. Ultrawidefield OCT (UWF-OCT) images were obtained from control subjects after consent was obtained.
Results: Three UWF-OCT images were used from healthy controls. We were able to generate color-inferenced images (see FIG. 2A above) that appeared similar to scanning laser ophthalmoscopy and retinal fundus photograph images, but using only OCT data.
Conclusions: Depth encoded signal obtained by volumetric OCT can be used to pseudo-colorize en face OCT images in the visible spectrum to simulate color fundus photography, which may aid in visualization of pathology and also have utility in medical education.

### Example 2

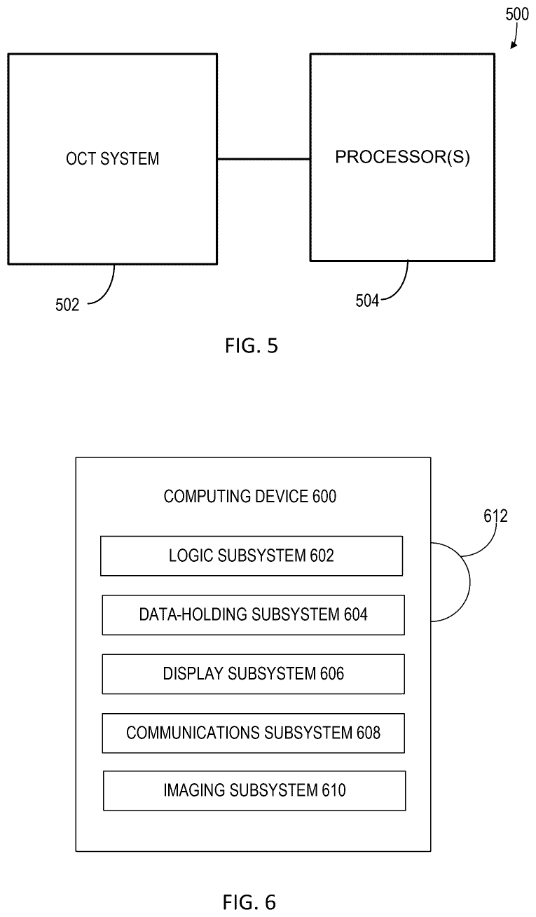

FIG. 5 schematically shows an exemplary system 500 for OCT image and OCT dataset processing in accordance with various embodiments. System 500 comprises an OCT system 502 configured to acquire an OCT image comprising OCT interferograms and one or more processors or computing systems 404 that are configured to implement the various processing routines described herein. OCT system 500 may comprise an OCT system suitable for wide-field OCT imaging applications.

In various embodiments, an OCT system may be adapted to allow an operator to perform various tasks. For example, an OCT system may be adapted to allow an operator to configure and/or launch various ones of the herein described methods. In some embodiments, an OCT system may be adapted to generate, or cause to be generated, reports of various information including, for example, reports of the results of scans run on a sample.

In embodiments of OCT systems comprising a display device, data and/or other information may be displayed for an operator. In embodiments, a display device may be adapted to receive an input (e.g., by a touch screen, actuation of an icon, manipulation of an input device such as a joystick or knob, etc.) and the input may, in some cases, be communicated (actively and/or passively) to one or more processors. In various embodiments, data and/or information may be displayed, and an operator may input information in response thereto.

In some embodiments, the methods and processes described herein may be tied to a computing system, including one or more computers. In particular, the methods and processes described herein, e.g., the simulation methods a segmentation-based methods to generate a color image from an OCT dataset described above, may be implemented as a computer application, computer service, computer API, computer library, and/or other computer program product.

FIG. 6 schematically shows a non-limiting computing device 600 that may perform one or more of the above-described methods and processes. For example, computing device 500 may represent a processor included in system 500 described above, and may be operatively coupled to, in communication with, or included in an OCT system or OCT image acquisition apparatus. Computing device 600 is shown in simplified form. It is to be understood that virtually any computer architecture may be used without departing from the scope of this disclosure. In different embodiments, computing device 600 may take the form of a microcomputer, an integrated computer circuit, printed circuit board (PCB), microchip, a mainframe computer, server computer, desktop computer, laptop computer, tablet computer, home entertainment computer, network computing device, mobile computing device, mobile communication device, gaming device, etc.

Computing device 600 includes a logic subsystem 602 and a data-holding subsystem 604. Computing device 600 may optionally include a display subsystem 606, a communication subsystem 608, an imaging subsystem 610, and/or other components not shown in FIG. 6. Computing device 600 may also optionally include user input devices such as manually actuated buttons, switches, keyboards, mice, game controllers, cameras, microphones, and/or touch screens, for example.

Logic subsystem 602 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. For example, the one or more processors may comprise physical circuitry programmed to perform various acts described herein. Additionally, or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 604 may include one or more physical, non-transitory, devices configured to hold data and/or instructions executable by the logic subsystem to implement the herein described methods and processes. When such methods and processes are implemented, the state of data-holding subsystem 604 may be transformed (e.g., to hold different data).

Data-holding subsystem 604 may include removable media and/or built-in devices. Data-holding subsystem 604 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 604 may include devices with one or more of the following characteristics: volatile, nonvolatile, dynamic, static, read/write, read-only, random access, sequential access, location addressable, file addressable, and content addressable. In some embodiments, logic subsystem 602 and data-holding subsystem 604 may be integrated into one or more common devices, such as an application specific integrated circuit or a system on a chip.

FIG. 6 also shows an aspect of the data-holding subsystem in the form of removable computer-readable storage media 612, which may be used to store and/or transfer data and/or instructions executable to implement the herein described methods and processes. Removable computer-readable storage media 612 may take the form of CDs, DVDs, HD-DVDs, Blu-Ray Discs, EEPROMs, flash memory cards, USB storage devices, and/or floppy disks, among others.

When included, display subsystem 606 may be used to present a visual representation of data held by data-holding subsystem 604. As the herein described methods and processes change the data held by the data-holding subsystem, and thus transform the state of the data-holding subsystem, the state of display subsystem 606 may likewise be transformed to visually represent changes in the underlying data. Display subsystem 606 may include one or more display devices utilizing virtually any type of technology. Such display devices may be combined with logic subsystem 602 and/or data-holding subsystem 604 in a shared enclosure, or such display devices may be peripheral display devices.

When included, communication subsystem 608 may be configured to communicatively couple computing device 600 with one or more other computing devices. Communication subsystem 508 may include wired and/or wireless communication devices compatible with one or more different communication protocols. As non-limiting examples, the communication subsystem may be configured for communication via a wireless telephone network, a wireless local area network, a wired local area network, a wireless wide area network, a wired wide area network, etc. In some embodiments, the communication subsystem may allow computing device 600 to send and/or receive messages to and/or from other devices via a network such as the Internet.

When included, imaging subsystem 610 may be used acquire and/or process any suitable image data from various sensors or imaging devices in communication with computing device 500. For example, imaging subsystem 610 may be configured to acquire OCT image data, e.g., interferograms, as part of an OCT system, e.g., OCT system 502 described above. Imaging subsystem 610 may be combined with logic subsystem 602 and/or data-holding subsystem 604 in a shared enclosure, or such imaging subsystems may comprise periphery imaging devices. Data received from the imaging subsystem may be held by data-holding subsystem 604 and/or removable computer-readable storage media 612, for example.

Skilled persons will appreciate that many changes may be made to the details of the above-described embodiments without departing from the underlying principles of the invention. The scope of the present invention should, therefore, be determined only by claimed inventions and equivalents thereof.

What is claimed is:

1. A method of generating a color image from an OCT dataset comprising:
obtaining an OCT dataset of a retina and choroid, the OCT dataset comprising a plurality of A-scans, each of the A-scans comprising a plurality of pixels extending depth-wise through the retina and each having an OCT signal value;
segmenting the pixels of the OCT dataset into a retinal slab and a choroidal slab;
initializing a blue channel, a green channel, and a red channel, each channel comprised of a plurality of pixels having correspondence to the plurality of A-scans of the OCT dataset;
for each of the blue channel, the green channel, and the red channel:
calculating, for each A-scan within the retinal and choroidal slabs, a total reflected signal by simulating passage of a photon packet stepwise though the A-scan, wherein the OCT signal values within the choroid slab are inverted when the red channel is simulated;
assigning the total reflected signal to the pixel corresponding to the A-scan for each of the blue channel, the green channel, and the red channel; and
merging the blue channel, green channel, and red channel, thereby generating a color image.

2. The method of claim 1, further comprising normalizing the pixels of the blue channel, the green channel, and the red channel.

3. The method of claim 1, wherein simulating passage of a photon packet stepwise though the A-scan comprises:
specifying, for the photon packet, an intensity value and a reflectivity parameter;
simulating a progressive attenuation of the intensity value due to reflection, scatter, and absorption as the photon packet proceeds stepwise through the A-scan; and
accumulating the progressive attenuation of the intensity value due to reflection as the photon packet proceeds stepwise through A-scan to calculate the total reflected signal.

4. The method of claim 3, wherein the green reflectivity parameter is smaller than the blue reflectivity parameter, and wherein the red reflectivity parameter is smaller than the green. reflectivity parameter.

5. A method of generating a color image from an OCT dataset of comprising:
obtaining the OCT dataset;
segmenting the OCT dataset into at least three slabs;
creating, from the at least three slabs, a set of en face projection images;
creating a blue channel, a green channel, and a red channel, wherein each of the blue channel, green channel, and red channel receives a weighted contribution from the set of en face projection images; and
merging the blue channel, the green channel, and the red channel, thereby generating a color image.

6. The method of claim 5, wherein each of the three or more segmented slabs comprises at least one of an anatomical region selected from the group of a retinal nerve fiber layer (RNFL), a retinal pigmented epithelium (RPE), a retina, a portion of a retina, a choroid, and a portion of a choroid.

7. The method of claim 5, further comprising normalizing the blue channel, the green channel, and the red channel.

8. A method of generating a color image from an OCT dataset comprising:
obtaining an OCT dataset of a retina and a choroid, the OCT dataset comprising plurality of OCT signal values, and the retina comprising an RNFL and an RPE;
segmenting OCT dataset into a first slab including the RNFL, a second slab including the RPE, and a third slab comprising the choroid;
inverting the OCT signal values within the third slab;
generating, from the first slab, a first normalized en face projection image;
populating a blue channel with the first normalized en face projection image;
generating, from the second slab, a second normalized en face projection image;
populating a green channel with the second normalized en face projection image;
generating, from the third slab, a third normalized en face projection image;
populating a red channel with the third normalized en face projection image; and
merging the blue channel, green channel, and red channel, thereby generating a color image.

9. The method of claim 8, wherein the first, second, and third en face projection images are calculated using mean intensity, maximum intensity, or summed intensity.

10. A method of generating a color image from an OCT dataset comprising:
obtaining an OCT dataset of a retina and a choroid;
generating, from the OCT dataset, a first projection image;
generating, from the OCT dataset, a second projection image;
populating each of a green channel and a red channel with a weighted contribution from the first projection image and the second projection image; and
merging the green channel and the red channel, thereby generating a color image.

11. The method of claim 10, wherein the weighted contribution from the first projection image and the second projection image is generated by:
populating the red channel with the first projection image; and
populating the green channel with a weighted sum of the first projection image and the second projection image.

12. The method of claim 11, further comprising populating a blue channel with a weighted contribution from the second projection image.

13. The method of claim 10, wherein the first projection image is a mean intensity projection image and the second projection is a maximum intensity projection image.

14. A system comprising:
an OCT system;
a logic subsystem; and
a data holding subsystem comprising non-transitory machine-readable instructions stored thereon that are executable by the logic subsystem to:
obtain an OCT dataset of a retina and choroid, the OCT dataset comprising a plurality of A-scans, each of the A-scans comprising a plurality of pixels extending depth-wise through the retina and each having an OCT signal value;
segment the pixels of the OCT dataset into a retinal slab and a choroidal slab;
initialize a blue channel, a green channel, and a red channel, each channel comprised of a plurality of pixels having correspondence to the plurality of A-scans of the OCT dataset;
for each of the blue channel, the green channel, and the red channel:
calculate, for each A-scan within the retinal and choroidal slabs, a total reflected signal by simulating passage of a photon packet stepwise though the A-scan, wherein the OCT signal values within the choroid slab are inverted when the red channel is simulated;
assign the total reflected signal to the pixel corresponding to the A-scan for each of the blue channel, the green channel, and the red channel; and
merge the blue channel, green channel, and red channel, thereby generating a color image.

15. The system of claim 14, further comprising instructions to normalize the pixels of the blue channel, the green channel, and the red channel.

16. The system of claim 14, wherein simulating passage of a photon packet stepwise though the A-scan comprises:
specifying, for the photon packet, an intensity value and a reflectivity parameter;
simulating a progressive attenuation of the intensity value due to reflection, scatter, and absorption as the photon packet proceeds stepwise through the A-scan; and
accumulating the progressive attenuation of the intensity value due to reflection as the photon packet proceeds stepwise through A-scan to calculate the total reflected signal.

17. The system of claim 16, wherein the green reflectivity parameter is smaller than the blue reflectivity parameter, and wherein the red reflectivity parameter is smaller than the green. reflectivity parameter.

18. A system comprising:
an OCT system;
a logic subsystem; and
a data holding subsystem comprising non-transitory machine-readable instructions stored thereon that are executable by the logic subsystem to:
obtain an OCT dataset;
segment the OCT dataset into at least three slabs;
create, from the at least three slabs, a set of en face projection images;
create a blue channel, a green channel, and a red channel, wherein each of the blue channel, green channel, and red channel receives a weighted contribution from the set of en face projection images; and
merge the blue, green, and red channels, thereby generating a color image.

19. The system of claim 18, wherein each of the three or more segmented slabs comprises at least one of an anatomical region selected from the group of a retinal nerve fiber layer (RNFL), a retinal pigmented epithelium (RPE), a retina, a portion of a retina, a choroid, and a portion of a choroid.

20. The system of claim 18, further comprising instructions to normalize the blue channel, the green channel, and the red channel.
